(19) United States
(12) Patent Application Publication
(10) Pub. No.: US 2009/0156398 A1
(43) Pub. Date: Jun. 18, 2009

(54) Fungicidal 5-alkyl-6-phenylpyrazolopyrimidin-7-ylamines

(75) Inventors: Jochen Dietz, Mannheim (DE); Wassilios Grammenos, Ludwigshafen (DE); Udo Hünger, Lambsheim (DE); Jan Klaas Lohmann, Ludwigshafen (DE); Jens Renner, Bad Dürkheim (DE); Joachim Rheinheimer, Ludwigshafen (DE)

Correspondence Address: BRINKS, HOFER, GILSON & LIONE, P.O. BOX 1340, MORRISVILLE, NC, 27560, US

(73) Assignee: BASF Aktiengesellschaft, Ludwigshafen (DE)

(21) Appl. No.: 11/996,780

(22) PCT Filed: Jul. 20, 2006
(86) PCT No.: PCT/EP06/64465

§ 371 Date: Jan. 25, 2008

(30) Foreign Application Priority Data
Jul. 27, 2005 (DE) 10 2005 035 689.3

(51) Int. Cl. A01C 1/06; C07D 487/04; A01N 43/90

(52) U.S. Cl. 504/100; 544/281; 514/259.31

(57) ABSTRACT

5-Alkyl-6-phenylpyrazolopyrimidin-7-ylamines of the formula (I) in which the substituents are defined as in the description, processes for preparing these compounds, compositions comprising them, and their use for controlling phytopathogenic fungi.

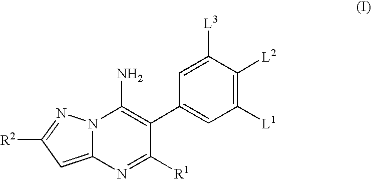

The present invention relates to 5-alkyl-6-phenylpyrazolopyrimidin-7-ylamines of the formula I,

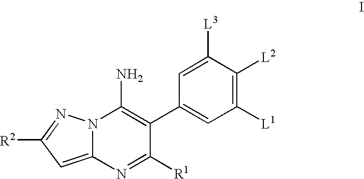

in which the substituents are as defined below:
L¹, L³ independently of one another are hydrogen, halogen, hydroxyl, mercapto, nitro, NR^{A}R^{B}, C₁-C₄-haloalkyl, C₁-C₁₀-alkyl, C₂-C₆-alkenyl, C₂-C₆-alkynyl, C₁-C₈-alkoxy, phenyl, phenoxy, phenylthio, benzyloxy or benzylthio;
   R^{A}, R^{B} are hydrogen or C₁-C₆-alkyl;
L² is hydrogen, halogen, hydroxyl, mercapto, nitro, NR^{A}R^{B}, C₁-C₄-haloalkyl, CH₂—C₁-C₆-alkyl, C₂-C₆-alkenyl, C₂-C₆-alkynyl, C₁-C₈-alkoxy, phenyl, phenoxy, phenylthio, benzyloxy or benzylthio;
   where two adjacent groups from the group consisting of L¹, L² and L³ together may be a C₁-C₄-alkylene, C₂-C₄-oxyalkylene, C₁-C₃-oxyalkyleneoxy or butadienyl group;
   where at least one group L¹, L² or L³ is not hydrogen and the groups L¹, L² or L³ are unsubstituted or substituted by one to four identical or different groups R^{a}:
   R^{a} is halogen, cyano, hydroxyl, mercapto, C₁-C₁₀-alkyl, C₁-C₁₀-haloalkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl or NR^{A}R^{B};
R¹ is ethyl or n-propyl;
R² is hydrogen, halogen, cyano, NR^{A}R^{B}, hydroxyl, mercapto, C₁-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkenyloxycarbonyl, phenyl, phenoxy, phenylthio, benzyloxy, benzylthio, C₁-C₆-alkyl-S(O)ₘ— or a five- or six-membered saturated, partially unsaturated or aromatic heterocycle which comprises one to four heteroatoms from the group consisting of O, N and S;
   m is 0, 1 or 2;
   where the cyclic groups in L¹, L², L³, R^{a} and/or R¹ are unsubstituted or substituted by one to four groups R^{b}:
   R^{b} is halogen, cyano, hydroxyl, mercapto, nitro, NR^{A}R^{B}, C₁-C₁₀-alkyl, C₁-C₆-haloalkyl, C₂-C₆-alkenyl, C₂-C₆-alkynyl, C₁-C₆-alkoxy or a five- or six-membered saturated, partially unsaturated or aromatic heterocycle which contains one to four heteroatoms from the group consisting of O, N and S which may be unsubstituted or substituted by one or more halogen and/or C₁-C₄-alkyl groups.

Moreover, the invention relates to processes for preparing these compounds, to compositions comprising them and to their use for controlling phytopathogenic harmful fungi.

Individual fungicidally active 5-methyl-6-phenylpyrazolopyrimidinylamines are known from EP-A 71 792. However, in many cases their activity is unsatisfactory. Based on this, it is an object of the present invention to provide compounds having improved activity and/or a widened activity spectrum.

We have found that this object is achieved by the compounds defined at the outset. Furthermore, we have found processes and intermediates for their preparation, compositions comprising them and methods for controlling harmful fungi using the compounds I.

The compounds of the formula I differ from the compounds known from EP-A 71 792 essentially by the substitution in positions 2 and 5 and/or the substitution of the phenyl ring in position 6 of the pyrazolopyrimidine skeleton.

Compared to the known compounds, the compounds of the formula I are more effective against harmful fungi.

The compounds according to the invention can be obtained by different routes. Advantageously, the compounds according to the invention are obtained by reaching substituted β-keto esters of the formula II with aminopyrazoles of the formula III to give 7-hydroxypyrazolopyrimidines of the formula IV. The groups L¹ to L³ and R¹ in the formulae II and IV are as defined for formula I and the group R in formula II is C₁-C₄-alkyl; for practical reasons, preference is given here to methyl, ethyl or propyl.

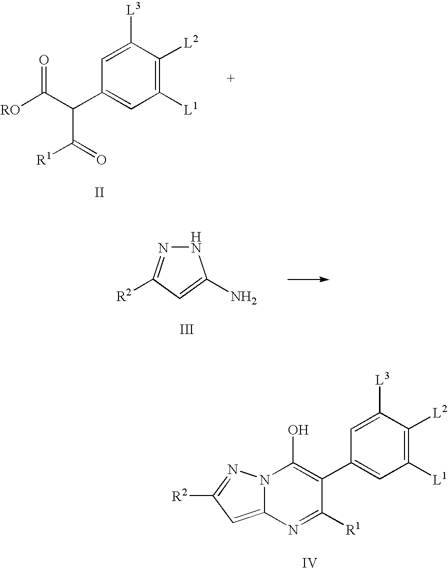

The reaction of the substituted β-keto esters of the formula II with the aminopyrazoles of the formula III can be carried out in the presence or absence of solvents. It is advantageous to use solvents to which the starting materials are substantially inert and in which they are completely or partially soluble. Suitable solvents are in particular alcohols, such as ethanol, propanols, butanols, glycols or glycol monoethers, diethylene glycols or their monoethers, aromatic hydrocarbons, such as toluene, benzene or mesitylene, amides, such as dimethylformamide, diethylformamide, dibutylformamide, N,N-dimethylacetamide, lower alkanoic acids, such as formic acid, acetic acid, propionic acid, or bases, such as alkali metal and alkaline earth metal hydroxides, alkali metal and alkaline earth metal oxides, alkali metal and alkaline earth metal hydrides, alkali metal amides, alkali metal and alkaline earth metal carbonates and also alkali metal bicarbonates, organometallic compounds, in particular alkali metal alkyls, alkyl-magnesium halides and also alkali metal and alkaline earth metal alkoxides and dimethoxymagnesium, moreover organic bases, for example tertiary amines, such as trimethylamine, triethylamine, triisopropylamine, tributylamine and N-methylpiperidine, N-methylmorpholine, pyridine, substituted pyridines, such as collidine, lutidine and 4-dimethylaminopyridine, and also bicyclic amines and mixtures of these solvents with water. Suitable catalysts are bases, as mentioned previously, or acids, such as sulfonic acids or mineral acids. With particular preference, the reaction is carried out without solvent or in chlorobenzene, xylene, dimethyl sulfoxide or N-methylpyrrolidone. Particularly preferred bases are tertiary amines, such as triisopropylamine, tributylamine, N-methylmorpholine or N-methylpiperidine. The temperatures are from 50 to 300° C., preferably from 50 to 180° C., if the reaction is carried out in solution [cf. EP-A 770 615; Adv. Het. Chem. 57 (1993), pp. 81ff].

The bases are generally employed in catalytic amounts; however, they can also be employed in equimolar amounts, in excess or, if appropriate, as solvent.

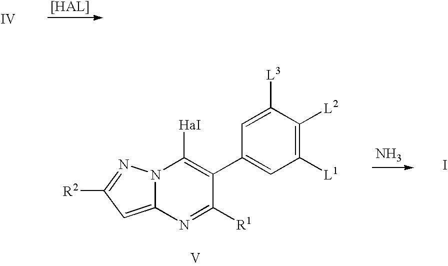

In most cases, the resulting condensates of the formula IV precipitate from the reaction solutions in pure form and, after washing with the same solvent or with water and subsequent drying, they are reacted with halogenating agents, in particular chlorinating or brominating agents, to give the compounds of the formula V in which Hal is chlorine or bromine, in particular chlorine. The reaction is preferably carried out using chlorinating agents such as phosphorus oxychloride, thionyl chloride or sulfonyl chloride at from 50° C. to 150° C., preferably in excess phosphorus oxytrichloride at reflux temperature. After evaporation of excess phosphorus oxytrichloride, the residue is treated with ice-water, if appropriate with addition of a water-immiscible solvent. In most cases, the chlorinated product isolated from the dried organic phase, if appropriate after evaporation of the inert solvent, is very pure and is subsequently reacted with ammonia in inert solvents at from 100° C. to 200° C. to give the 7-aminopyrazolopyrimidines. This reaction is preferably carried out using a 1- to 10-molar excess of ammonia, under a pressure of from 1 to 100 bar.

The novel pyrazolopyrimidine-7-ylamines are, if appropriate after evaporation of the solvent, isolated as crystalline compounds, by digestion in water.

The β-keto esters of the formula II can be prepared as described in Organic Synthesis Coll. Vol. 1, p. 248, and/or they are commercially available.

Alternatively, the novel compounds of the formula I can be obtained by reacting substituted acyl cyanides of the formula VI, in which L¹ to L³ are as defined above, with aminopyrazoles of the formula III.

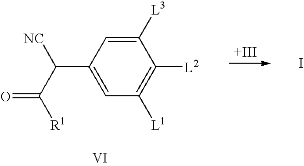

The reaction can be carried out in the presence or absence of solvents. It is advantageous to use solvents to which the starting materials are substantially inert and in which they are completely or partially soluble. Suitable solvents are in particular alcohols, such as ethanol, propanols, butanols, glycols or glycol monoethers, diethylene glycols or their monoethers, aromatic hydrocarbons, such as toluene, benzene or mesitylene, amides, such as dimethylformamide, diethylformamide, dibutylformamide, N,N-dimethylacetamide, lower alkanoic acids, such as formic acid, acetic acid, propionic acid, or bases, such as those mentioned above, and mixtures of these solvents with water. The reaction temperatures are from 50 to 300° C., preferably from 50 to 150° C., if the reaction is carried out in solution.

The novel pyrazolopyrimidine-7-ylamines are, if appropriate after evaporation of the solvent or dilution with water, isolated as crystalline compounds.

Some of the substituted alkyl cyanides of the formula VI required for preparing the pyrazolopyrimidine-7-ylamines are known, or they can be prepared by known methods from alkyl cyanides and carboxylic esters using strong bases, for example alkali metal hydrides, alkali metal alkoxides, alkali metal amides or metal alkyls [cf.: J. Amer. Chem. Soc. 73, (1951), p. 3766].

If individual compounds I cannot be obtained by the routes described above, they can be prepared by derivatization of other compounds I.

If the synthesis yields mixtures of isomers, a separation is generally not necessarily required since in some cases the individual isomers can be interconverted during work-up for use or during use (for example under the action of light, acids or bases). Such conversions may also take place after use, for example, in the case of treatment of plants, in the treated plants, or in the harmful fungus to be controlled.

In the definitions of symbols given in the formulae above, collective terms were used which are generally representative of the following substituents:

halogen: fluorine, chlorine, bromine and iodine;

alkyl: saturated straight-chain or mono- or dibranched hydrocarbon radicals having 1 to 4, 6 or 8 carbon atoms, for example C₁-C₆-alkyl such as methyl, ethyl, propyl, 1-methylethyl, butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: an alkyl group as mentioned above in which some or all of the hydrogen atoms may be replaced by halogen atoms as mentioned above; in particular chloromethyl, bromomethyl, dichloromethyl, trichloromethyl, fluoromethyl, difluoromethyl, trifluoromethyl, chlorofluoromethyl, dichlorofluoromethyl, chlorodifluoromethyl;

cycloalkyl: mono- or bicyclic saturated hydrocarbon groups having 3 to 6 carbon ring members, such as cyclopropyl, cyclobutyl, cyclopentyl and cyclohexyl;

alkoxyalkyl: a saturated straight-chain or mono-, di- or tribranched hydrocarbon chain which is interrupted by an oxygen atom, for example C₅-C₁₂-alkoxyalkyl: a hydrocarbon chain as described above having 5 to 12 carbon atoms which may be interrupted by an oxygen atom in any position, such as propoxyethyl, butoxyethyl, pentoxyethyl, hexyloxyethyl, heptyloxyethyl, octyloxyethyl, nonyloxyethyl, 3-(3-ethylhexyloxy)ethyl, 3-(2,4,4-trimethylpentyloxy)ethyl, 3-(1-ethyl-3-methylbutoxy)ethyl, ethoxypropyl, propoxypropyl, butoxypropyl, pentoxypropyl, hexyloxypropyl, heptyloxypropyl, octyloxypropyl, nonyloxypropyl, 3-(3-ethylhexyloxy)propyl, 3-(2,4,4-trimethylpentyloxy)propyl, 3-(1-ethyl-3-methylbutoxy)propyl, ethoxybutyl, propoxybutyl, butoxybutyl, pentoxybutyl, hexyloxybutyl, heptyloxybutyl, octyloxybutyl, nonyloxybutyl, 3-(3-ethylhexyloxy)butyl, 3-(2,4,4-trimethylpentyloxy)butyl, 3-(1-ethyl-3-methylbutoxy)butyl, methoxypentyl, ethoxypentyl, propoxypentyl, butoxypentyl, pentoxypentyl, hexyloxypentyl, heptyloxypentyl, 3-(3-methylhexyloxy)pentyl, 3-(2,4-dimethylpentyloxy)pentyl, 3-(1-ethyl-3-methylbutoxy)pentyl;

alkenyl: unsaturated straight-chain or branched hydrocarbon radicals having 2 to 4, 6, 8 or 10 carbon atoms and one or two double bonds in any position, for example C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

alkynyl: straight-chain or branched hydrocarbon groups having 2 to 4 or 6 carbon atoms and one or two triple bonds in any position, for example C₂-C₆-alkynyl such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

alkylene: divalent unbranched chains, preferably of 3 to 5 CH₂ groups, for example CH₂, CH₂CH₂, CH₂CH₂CH₂, CH₂CH₂CH₂CH₂ and CH₂CH₂CH₂CH₂CH₂;

oxyalkylene: divalent unbranched chains of 2 to 4 CH₂ groups where one valency is attached to the skeleton via an oxygen atom, for example OCH₂CH₂, OCH₂CH₂CH₂ and OCH₂CH₂CH₂CH₂;

oxyalkyleneoxy: divalent unbranched chains of 1 to 3 CH₂ groups where both valencies are attached to the skeleton via an oxygen atom, for example OCH₂O, OCH₂CH₂O and OCH₂CH₂CH₂O.

The scope of the present invention includes the (R)- and (S)-isomers and the racemates of compounds of the formula I having chiral centers.

With a view to the intended use of the pyrazolopyrimidinylamines of the formula I, particular preference is given to the following meanings of the substituents, in each case on their own or in combination:

Preference is given to compounds I in which the 6-phenyl group is substituted by one to three halogen or CH₂—C₁-C₄-alkyl groups.

A preferred embodiment of the compounds of the formula I are those in which group R^{a} is absent.

A further preferred embodiment relates to compounds of the formula I in which L¹ and L³ are hydrogen. Particular preference is given to those compounds in which L² is halogen or alkyl, in particular alkyl.

A further preferred embodiment relates to compounds of the formula I in which L² and L³ are hydrogen. Particular preference is given to those compounds in which L¹ is halogen or alkyl.

A further preferred embodiment relates to compounds of the formula I in which L¹ and L² are not hydrogen and L³ is hydrogen. Particular preference is given to those compounds in which L¹ and L² are halogen.

A further preferred embodiment relates to compounds of the formula I in which the 6-phenyl group is substituted by one to three halogen, cyano, hydroxyl, mercapto, nitro, NR^{A}R^{B}, C₁-C₁₀-alkyl, C₁-C₆-haloalkyl, C₂-C₆-alkenyl, C₂-C₆-alkynyl and C₁-C₆-alkoxy groups. With particular preference, the phenyl group carries two, in particular one, substituent(s).

One embodiment relates to compounds of the formula I in which R¹ is ethyl.

A further embodiment relates to compounds of the formula I in which R¹ is n-propyl.

A further preferred embodiment relates to compounds of the formula I in which R² is not hydrogen.

In a further embodiment of the compounds I, R² is NH₂ or C₁-C₄-alkyl, preferably C₁-C₂-alkyl or NH₂, in particular methyl.

Particular preference is given to compounds of the formula I in which L¹ is cyano, hydroxyl, mercapto, nitro, NR^{A}R^{B}, C₁-C₆-alkyl, halomethyl or C₁-C₂-alkoxy.

With respect to their use, particular preference is given to the compounds I compiled in the tables below. Moreover, the groups mentioned for a substituent in the tables are, by themselves and independently of the combination in which they are mentioned, a particularly preferred embodiment of the substituent in question.

Table 1

Compounds of the formula I in which R¹ is ethyl, R² is methyl and the combination of L¹, L² and L³ for a compound corresponds in each case to one row of Table A

Table 2

Compounds of the formula I in which R¹ is ethyl, R² is amino and the combination of L¹, L² and L³ for a compound corresponds in each case to one row of Table A

Table 3

Compounds of the formula I in which R¹ is n-propyl, R² is methyl and the combination of L¹, L² and L³ for a compound corresponds in each case to one row of Table A

Table 4

Compounds of the formula I in which R¹ is n-propyl, R² is amino and the combination of L¹, L² and L³ for a compound corresponds in each case to one row of Table A

TABLE A

| No. | L¹ | L² | L³ |
|---|---|---|---|
| A-1 | CH₃ | H | H |
| A-2 | CH₃ | H | CH₃ |
| A-3 | CH₂CH₃ | H | H |
| A-4 | H | CH₂CH₃ | H |
| A-5 | CH₂CH₃ | CH₂CH₃ | H |
| A-6 | CH₂CH₃ | H | CH₂CH₃ |
| A-7 | CH₂CH₂CH₃ | H | H |
| A-8 | H | CH₂CH₂CH₃ | H |
| A-9 | CH₂CH₂CH₃ | CH₂CH₂CH₃ | H |
| A-10 | CH₂CH₂CH₃ | H | CH₂CH₂CH₃ |
| A-11 | CH(CH₃)₂ | H | H |
| A-12 | CH(CH₃)₂ | H | CH(CH₃)₂ |
| A-13 | CH₂CH₂CH₂CH₃ | H | H |
| A-14 | H | CH₂CH₂CH₂CH₃ | H |
| A-15 | CH₂CH₂CH₂CH₃ | CH₂CH₂CH₂CH₃ | H |
| A-16 | CH₂CH₂CH2CH₃ | H | CH₂CH₂CH₂CH₃ |
| A-17 | CH₂CH(CH₃)₂ | H | H |
| A-18 | H | CH₂CH(CH₃)₂ | H |
| A-19 | CH₂CH(CH₃)2 | H | CH₂CH(CH₃)₂ |
| A-20 | C(CH₃)₃ | H | H |
| A-21 | (CH₂)₄CH₃ | H | H |
| A-22 | H | (CH₂)₄CH₃ | H |
| A-23 | (CH₂)₄CH₃ | (CH₂)₄CH₃ | H |
| A-24 | (CH₂)₄CH₃ | H | (CH₂)₄CH₃ |
| A-25 | CH₂CH₂CH(CH₃)₂ | H | H |
| A-26 | H | CH₂CH₂CH(CH₃)₂ | H |
| A-27 | CH₂CH₂CH(CH₃)₂ | H | CH₂CH₂CH(CH₃)₂ |
| A-28 | CH₂CH₂CH(CH₃)₂ | H | H |
| A-29 | CH₂CH(CH₃)CH₂CH₃ | H | H |
| A-30 | H | CH₂CH(CH₃)CH₂CH₃ | H |
| A-31 | CH₂CH(CH₃)CH₂CH₃ | H | CH₂CH(CH₃)CH₂CH₃ |
| A-32 | CH₂C(CH₃)₃ | H | H |
| A-33 | H | CH₂C(CH₃)₃ | H |
| A-34 | CH₂C(CH₃)₃ | H | CH₂C(CH₃)₃ |
| A-35 | (CH₂)₅CH₃ | H | H |
| A-36 | H | (CH₂)₅CH₃ | H |
| A-37 | (CH₂)₅CH₃ | (CH₂)₅CH₃ | H |
| A-38 | (CH₂)₅CH₃ | H | (CH₂)₅CH₃ |
| A-39 | CH₂CH(CH₂CH₃)₂ | H | H |
| A-40 | H | CH₂CH(CH₂CH₃)₂ | H |
| A-41 | CH₂CH(CH₂CH₃)₂ | H | CH₂CH(CH₂CH₃)₂ |
| A-42 | CH₂C(CH₃)₂CH₂CH₃ | H | H |
| A-43 | H | CH₂C(CH₃)₂CH₂CH₃ | H |
| A-44 | CH₂C(CH₃)₂CH₂CH₃ | H | CH₂C(CH₃)₂CH₂CH₃ |
| A-45 | CH₂C(CH₂CH₃)₂CH₃ | H | H |
| A-46 | H | CH₂C(CH₂CH₃)₂CH₃ | H |
| A-47 | CH₂C(CH₂CH₃)₂CH₃ | H | CH₂C(CH₂CH₃)₂CH₃ |
| A-48 | Cl | H | H |
| A-49 | H | Cl | H |
| A-50 | Cl | Cl | H |
| A-51 | Cl | H | Cl |
| A-52 | Cl | Cl | Cl |
| A-53 | F | H | H |
| A-54 | H | F | H |
| A-55 | F | F | H |
| A-56 | F | H | F |
| A-57 | F | F | F |
| A-58 | Br | H | H |
| A-59 | H | Br | H |
| A-60 | Br | Br | H |
| A-61 | Br | H | Br |
| A-62 | Br | Br | Br |
| A-63 | CHF₂ | H | H |
| A-64 | H | CHF₂ | H |
| A-65 | CHF₂ | H | CHF₂ |
| A-66 | CHF₂ | CHF₂ | H |
| A-67 | CF₃ | H | H |
| A-68 | H | CF₃ | H |
| A-69 | CHF₂ | H | CF₃ |
| A-70 | CF₃ | CF₃ | H |
| A-71 | CH═CH₂ | H | H |
| A-72 | H | CH═CH₂ | H |
| A-73 | CH═CH₂ | CH═CH₂ | H |
| A-74 | CH═CH₂ | H | CH═CH₂ |
| A-75 | CH₂CH═CH₂ | H | H |
| A-76 | H | CH₂CH═CH₂ | H |
| A-77 | CH₂CH═CH₂ | CH₂CH═CH₂ | H |
| A-78 | CH₂CH═CH₂ | H | CH₂CH═CH₂ |
| A-79 | C≡CH | H | H |
| A-80 | H | C≡CH | H |
| A-81 | C≡CH | H | C≡CH |
| A-82 | CH₂C≡CH | H | H |
| A-83 | H | CH₂C≡CH | H |
| A-84 | CH₂C≡CH | H | CH₂C≡CH |
| A-85 | OCH₃ | H | H |
| A-86 | H | OCH₃ | H |
| A-87 | OCH₃ | H | OCH₃ |
| A-88 | OCH₂CH₃ | H | H |
| A-89 | H | OCH₂CH₃ | H |
| A-90 | OCH₂CH₃ | H | OCH₂CH₃ |
| A-91 | OCH₂CH₂CH₃ | H | H |
| A-92 | H | OCH₂CH₂CH₃ | H |
| A-93 | OCH₂CH₂CH₃ | H | OCH₂CH₂CH₃ |
| A-94 | OCH(CH₃)₂ | H | H |
| A-95 | H | OCH(CH₃)₂ | H |
| A-96 | OCH(CH₃)₂ | H | OCH(CH₃)₂ |
| A-97 | O(CH₂)₃CH₃ | H | H |
| A-98 | H | O(CH₂)₃CH₃ | H |
| A-99 | O(CH₂)₃CH₃ | H | O(CH₂)₃CH₃ |
| A-100 | OCH₂CH(CH₃)₂ | H | H |
| A-101 | H | OCH₂CH(CH₃)₂ | H |
| A-102 | OCH₂CH(CH₃)₂ | H | OCH₂CH(CH₃)₂ |
| A-103 | OC(CH₃)₃ | H | H |
| A-104 | H | OC(CH₃)₃ | H |
| A-105 | OC(CH₃)₃ | H | OC(CH₃)₃ |
| A-106 | O(CH₂)₄CH₃ | H | H |
| A-107 | H | O(CH₂)₄CH₃ | H |
| A-108 | O(CH₂)₄CH₃ | H | O(CH₂)₄CH₃ |
| A-109 | OCH₂CH(CH₃)CH₂CH₃ | H | H |
| A-110 | H | OCH₂CH(CH₃)CH₂CH₃ | H |
| A-111 | OCH₂CH(CH₃)CH₂CH₃ | H | OCH₂CH(CH₃)CH₂CH₃ |
| A-112 | OCH₂C(CH₃)₃ | H | H |
| A-113 | H | OCH₂C(CH₃)₃ | H |
| A-114 | OCH₂C(CH₃)₃ | H | OCH₂C(CH₃)₃ |
| A-115 | O(CH₂)₅CH₃ | H | H |
| A-116 | H | O(CH₂)₅CH₃ | H |
| A-117 | O(CH₂)₅CH₃ | H | O(CH₂)₅CH₃ |
| A-118 | OCH₂CH(CH₂CH₃)₂ | H | H |
| A-119 | H | OCH₂CH(CH₂CH₃)₂ | H |
| A-120 | OCH₂CH(CH₂CH₃)₂ | H | OCH₂CH(CH₂CH₃)₂ |
| A-121 | C₆H₅ | H | H |
| A-122 | H | C₆H₅ | H |
| A-123 | OC₆H₅ | H | H |
| A-124 | H | OC₆H₅ | H |
| A-125 | SC₆H₅ | H | H |
| A-126 | H | SC₆H₅ | H |
| A-127 | CH₂C₆H₅ | H | H |
| A-128 | H | CH₂C₆H₅ | H |
| A-129 | OCH₂C₆H₅ | H | H |
| A-130 | H | OCH₂C₆H₅ | H |
| A-131 | SCH₂C₆H₅ | H | H |
| A-132 | H | SCH₂C₆H₅ | H |

The compounds I are suitable for use as fungicides. They are distinguished by excellent activity against a broad spectrum of phytopathogenic fungi from the classes of the Ascomycetes, Deuteromycetes, Peronosporomycetes (syn. Oomycetes) and Basidiomycetes, in particular from the class of the Peronosporomycetes. Some of them are systemically active and can be used in crop protection as foliar fungicides, as fungicides for seed dressing and as soil fungicides.

They are particularly important in the control of a large number of fungi on various crop plants, such as wheat, rye, barley, oats, rice, corn, grass, bananas, cotton, soybeans, coffee, sugar cane, grapevines, fruit and ornamental plants and vegetables, such as cucumbers, beans, tomatoes, potatoes and cucurbits, and also the seeds of these plants.

They are especially suitable for controlling the following plant diseases:
Alternaria species on vegetables, oilseed rape, sugar beet and fruit and rice, such as, for example, A. solani or A. alternata on potatoes and tomatoes;
Aphanomyces species on sugar beet and vegetables;
Ascochyta species on cereals and vegetables;
Bipolaris and Drechslera species on corn, cereals, rice and lawns, such as, for example, D. maydis on corn;
Blumeria graminis (powdery mildew) on cereals;
Botrytis cinerea (gray mold) on strawberries, vegetables, flowers and grapevines;
Bremia lactucae on lettuce;
Cercospora species on corn, soybeans, rice and sugar beet;
Cochliobolus species on corn, cereals, rice, such as, for example, Cochliobolus sativus on cereals, Cochliobolus miyabeanus on rice;
Colletotricum species on soybeans and cotton;
Drechslera species, Pyrenophora species on corn, cereals, rice and lawns, such as, for example, D. teres on barley or D. tritici-repentis on wheat;
Esca on grapevines, caused by Phaeoacremonium chlamydosporium, Ph. Aleophilum and Formitipora punctata (syn. Phellinus punctatus);
Exserohilum species on corn;
Erysiphe cichoracearum and Sphaerotheca fuliginea on cucumbers;
Fusarium and Verticillium species on various plants, such as, for example, F. graminearum or F. culmorum on cereals or F. oxysporum on a multitude of plants, such as, for example, tomatoes;
Gaeumanomyces graminis on cereals;
Gibberella species on cereals and rice (for example Gibberella fujikuroi on rice);
Grainstaining complex on rice;
Helminthosporium species on corn and rice;
Michrodochium nivale on cereals;
Mycosphaerella species on cereals, bananas and groundnuts, such as, for example, M. graminicola on wheat or M. fijiensis on bananas;
Peronospora species on cabbage and bulbous plants, such as, for example, P. brassicae on cabbage or P. destructor on onions;
Phakopsara pachyrhizi and Phakopsara meibomiae on soybeans;
Phomopsis species on soybeans and sunflowers;
Phytophthora infestans on potatoes and tomatoes;
Phytophthora species on various plants, such as, for example, P. capsici on bell pepper;
Plasmopara viticola on grapevines;
Podosphaera leucotricha on apples;
Pseudocercosporella herpotrichoides on cereals;
Pseudoperonospora on various plants, such as, for example, P. cubensis on cucumber or P. humili on hops;
Puccinia species on various plants, such as, for example, P. triticina, P. striformins, P. hordei or P. graminis on cereals or P. asparagi on asparagus;
Pyricularia oryzae, Corticium sasakii, Sarocladium oryzae, S. attenuatum, Entyloma oryzae on rice;
Pyricularia grisea on lawns and cereals;
Pythium spp. on lawns, rice, corn, cotton, oilseed rape, sunflowers, sugar beet, vegetables and other plants, such as, for example, P. ultiumum on various plants, P. aphanidermatum on lawns;
Rhizoctonia species on cotton, rice, potatoes, lawns, corn, oilseed rape, potatoes, sugar beet, vegetables and on various plants, such as, for example, R. solani on beet and various plants;
Rhynchosporium secalis on barley, rye and triticale;
Sclerotinia species on oilseed rape and sunflowers;
Septoria tritici and Stagonospora nodorum on wheat;
Erysiphe (syn. Uncinula) necator on grapevines;
Setospaeria species on corn and lawns;
Sphacelotheca reilinia on corn;
Thievaliopsis species on soybeans and cotton;
Tilletia species on cereals;
Ustilago species on cereals, corn and sugar cane, such as, for example, U. maydis on corn;
Venturia species (scab) on apples and pears, such as, for example, V. inaequalis on apples.

The compounds I are furthermore suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise from 0.1 to 95%, preferably from 0.5 to 90%, by weight of active compound.

When employed in plant protection, the amounts applied are, depending on the kind of effect desired, from 0.01 to 2.0 kg of active compound per ha.

In seed treatment, for example by dusting, coating or drenching seed, amounts of active compound of from 1 to 1000 g/100 kg, preferably from 5 to 100 g/100 kg, of seed are generally necessary.

When used in the protection of materials or stored products, the amount of active compound applied depends on the kind of application area and on the desired effect. Amounts customarily applied in the protection of materials are, for example, from 0.001 g to 2 kg, preferably from 0.005 g to 1 kg, of active compound per cubic meter of treated material.

The compounds of the formula I can be present in different crystal modifications which may differ in their biological activity. They also form part of the subject matter of the present invention.

The compounds I can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner, for example by extending the active compound with solvents and/or carriers, if desired using emulsifiers and dispersants. Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

Formulations for the treatment of seed may additionally comprise binders and/or gelling agents and, if appropriate, colorants.

Binders may be added to increase the adhesion of the active compounds on the seed after the treatment. Suitable binders are, for example, EO/PO block copolymer surfactants, but also polyvinyl alcohols, polyvinylpyrrolidones, polyacrylates, polymethacrylates, polybutenes, polyisobutylenes, polystyrenes, polyethylenamines, polyethylenamides, polyethylenimines (Lupasol®, Polymin®), polyethers, polyurethanes, polyvinyl acetates, tylose and copolymers of these polymers. A suitable gelling agent is, for example, carrageen (Satiagel®).

In general, the formulations comprise from 0.01 to 95% by weight, preferably from 0.1 to 90% by weight, of the active compound. The active compounds are employed in a purity of from 90% to 100%, preferably from 95% to 100% (according to NMR spectrum).

The concentrations of active compound in the ready-for-use preparations can be varied within relatively wide ranges. In general, they are from 0.0001 to 10%, preferably from 0.01 to 1%.

The active compounds can also be used with great success in the ultra-low volume (ULV) process, it being possible to apply formulations with more than 95% by weight of active compound or even the active compound without additives.

For the treatment of seed, the formulations in question give, after two-to-tenfold dilution, active compound concentrations of from 0.01 to 60% by weight, preferably from 0.1 to 40% by weight, in the ready-for-use preparations.

The following are examples of formulations according to the invention:
1. Products for dilution with water

A Water-Soluble Concentrates (SL, LS)

10 parts by weight of a compound according to the invention are dissolved in 90 parts by weight of water or in a water-soluble solvent. As an alternative, wetters or other auxiliaries are added. The active compound dissolves upon dilution with water. In this way, a formulation having a content of 10% by weight of active compound is obtained.

B Dispersible Concentrates (DC)

20 parts by weight of a compound according to the invention are dissolved in 70 parts by weight of cyclohexanone with addition of 10 parts by weight of a dispersant, for example polyvinylpyrrolidone. Dilution with water gives a dispersion. The active compound content is 20% by weight

C Emulsifiable Concentrates (EC)

15 parts by weight of a compound according to the invention are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

D Emulsions (EW, EO, ES)

25 parts by weight of a compound according to the invention are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (e.g. Ultraturrax) and made into a homogeneous emulsion. Dilution with water gives an emulsion. The formulation has an active compound content of 25% by weight.

E Suspensions (SC, OD, FS)

In an agitated ball mill, 20 parts by weight of a compound according to the invention are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

F Water-Dispersible Granules and Water-Soluble Granules (WG, SG)

50 parts by weight of a compound according to the invention are ground finely with addition of 50 parts by weight of dispersants and wetters and prepared as water-dispersible or water-soluble granules by means of technical appliances (for example extrusion, spray tower, fluidized bed). Dilution with water gives a stable dispersion or solution of the active compound. The formulation has an active compound content of 50% by weight.

G Water-Dispersible Powders and Water-Soluble Powders (WP, SP, SS, WS)

75 parts by weight of a compound according to the invention are ground in a rotor-stator mill with addition of 25 parts by weight of dispersants, wetters and silica gel. Dilution with water gives a stable dispersion or solution of the active compound. The active compound content of the formulation is 75% by weight.

H Gel Formulations

In a ball mill, 20 parts by weight of a compound according to the invention, 10 parts by weight of dispersant, 1 part by weight of gelling agent and 70 parts by weight of water or an organic solvent are ground to give a fine suspension. On dilution with water, a stable suspension having an active compound content of 20% by weight is obtained.
2. Products to be Applied Undiluted

I Dustable Powders (DP, DS)

5 parts by weight of a compound according to the invention are ground finely and mixed intimately with 95 parts by weight of finely divided kaolin. This gives a dustable product having an active compound content of 5% by weight.

J Granules (GR, FG, GG, MG)

0.5 part by weight of a compound according to the invention is ground finely and associated with 99.5 parts by weight of carriers. Current methods are extrusion, spray-drying or the fluidized bed. This gives granules to be applied undiluted having an active compound content of 0.5% by weight.

K ULV Solutions (UL)

10 parts by weight of a compound according to the invention are dissolved in 90 parts by weight of an organic solvent, for example xylene. This gives a product to be applied undiluted having an active compound content of 10% by weight.

For seed treatment, use is usually made of water-soluble concentrates (LS), suspensions (FS), dustable powders (DS), water-dispersible and water-soluble powders (WS, SS), emulsions (ES), emulsifiable concentrates (EC) and gel formulations (GF). These formulations can be applied to the seed in undiluted form or, preferably, diluted. Application can be carried out prior to sowing.

Preference is given to using FS formulations for seed treatment. Usually, such formulations comprise from 1 to 800 g of active compound/I, from 1 to 200 g of surfactants/I, from 0 to 200 g of antifreeze agents/I, from 0 to 400 g of binder/I, from 0 to 200 g of colorants/I and solvents, preferably water.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds according to the invention.

Aqueous use forms can be prepared from emulsion concentrates, pastes or wettable powders (wettable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

Oils of various types, wetters, adjuvants, herbicides, fungicides, other pesticides, or bactericides may be added to the active compounds, even, if appropriate, not until immediately prior to use (tank mix). These agents may be admixed with the compositions according to the invention in a weight ratio of from 1:100 to 100:1, preferably from 1:10 to 10:1.

Suitable adjuvants in this sense are in particular: organically modified polysiloxanes, for example Break Thru S 240®; alcohol alkoxylates, for example Atplus 245®, Atplus MBA 1303®, Plurafac LF 300® and Lutensol ON 30®; EO/PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention can, in the application form as fungicides, also be present together with other active compounds, for example with herbicides, insecticides, growth regulators, fungicides or also with fertilizers. By mixing the compounds (I) or the compositions comprising them with one or more further active compounds, in particular fungicides, it is in many cases possible to broaden the activity spectrum or to prevent the development of resistance. In many cases, synergistic effects are obtained.

The following list of fungicides, in conjunction with which the compounds according to the invention can be used, is intended to illustrate the possible combinations but does not limit them:

Strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethylphenyloxymethylene)phenyl)-3-methoxyacrylate;

Carboxamides
carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(3′,4′-dichloro-5-fluorobiphenyl-2-yl)-3-difluoromethyl-1-methylpyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide;

Azoles
triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;

Nitrogenous Heterocyclyl Compounds
pyridines: fluazinam, pyrifenox, 3-[5-(4-chlorophenyl)-2,3-dimethylisoxazolidin-3-y]-pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-S-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchroman-4-one, N,N-dimethyl-3-(3-bromo-6-fluoro-2-methylindole-1-sulfonyl)-[1,2,4]triazole-1-sulfonamide;

Carbamates and Dithiocarbamates
dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;

Other Fungicides
guanidines: dodine, iminoctadine, guazatine;
antibiotics: kasugamycin, polyoxins, streptomycin, validamycin A;
organometallic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate-methyl, chlorothalonil, dichlorfluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

### SYNTHESIS EXAMPLES

The procedure described in the following synthesis example was used to prepare further compounds I by appropriate modification of the starting materials. The compounds obtained in this manner are listed in the table below, together with physical data.

### Synthesis of 2-methyl-5-ethyl-6-(p-ethylphenyl)-7-aminopyrazolopyrimidine [I-1]

A suspension of 0.20 g (1 mmol) of 1-(p-ethylphenyl)-2-oxopropane-1-nitrile, 0.1 g (1 mmol) of 3-amino-5-methyl-1,2-pyrazole and 0.04 g (0.2 mmol) of p-toluenesulfonic acid in 2.5 ml of mesitylene was heated on a water separator at 160° C. for 1.5 hours. The mesitylene was then distilled off, and the residue was taken up in dichloromethane. After drying and removal of the solvent by distillation, the residue was recrystallized from diisopropyl ether. This gave 0.15 g of the title compound in the form of colorless crystals.

| | | | | | | phys. data |
|---|---|---|---|---|---|---|
| | | | | | | (m.p. [° C.]; |
| No. | R² | L¹ | L² | L³ | R¹ | ¹H-NMR δ [ppm] |
| I-1 | CH₃ | H | C₂H₅ | H | CH₂CH₃ | 155-156 |
| I-2 | CH₃ | CF₃ | H | H | CH₂CH₃ | 158-159 |
| I-3 | CH₃ | F | — | H | CH₂CH₂CH₃ | 6.1 (s); 6.7 (s); |
| | | | | | | 7.1-7.25 (m); |
| | | | | | | 7.5 (m) |
| I-4 | CH₃ | — | Cl | H | CH₂CH₂CH₃ | 0.7 (t); 1.5 (m); |
| | | | | | | 2.3 (m); 2.35 (s); |
| | | | | | | 6.1 (s); 6.7 (s); |
| | | | | | | 7.3-7.55 (m) |
| I-5 | CH₃ | — | F | H | CH₂CH₂CH₃ | 0.7 (t); 1.5 (m); |
| | | | | | | 2.3 (m); 2.35 (s); |
| | | | | | | 6.1 (s); 6.65 (s); |
| | | | | | | 7.2-7.4 (m) |

### Use Examples

The fungicidal effect of the compounds according to the invention was demonstrated by the following tests:

The active compounds were formulated separately as a stock solution having a concentration of 10 000 ppm in DMSO.

### Use Example 1

### Activity Against the Late Blight Pathogen Phytophthora infestans in the Microtiter Test

The stock solution is pipetted onto a microtiter plate (MTP) and diluted to the stated active compound concentration using a pea juice-based aqueous nutrient medium for fungi. An aqueous zoospore suspension of Phytophthora infestans was then added. The plates were placed in a water vapor-saturated chamber at temperatures of 18° C. Using an absorption photometer, the MTPs were measured at 405 nm on day 7 after the inoculation.

The measured parameters were compared to the growth of the active compound-free control variant and the fungus- and active compound-free blank value to determine the relative growth in % of the pathogens in the individual active compounds.

In this test, the growth of the pathogens was inhibited completely by 500 ppm of the active compound I-1 or I-2.

### Use Example No. 2

### Activity Against the Rice Blast Pathogen Pyricularia oryzae in the Microtiter Test

The stock solution is pipetted onto a microtiter plate (MTP) and diluted to the stated active compound concentration using a malt-based aqueous nutrient medium for fungi. An aqueous spore suspension of Pyricularia oryzae was then added. The plates were placed in a water vapor-saturated chamber at temperatures of 18° C. Using an absorption photometer, the MTPs were measured at 405 nm on day 7 after the inoculation.

The measured parameters were compared to the growth of the active compound-free control variant and the fungus- and active compound-free blank value to determine the relative growth in % of the pathogens in the individual active compounds.

In this test, the growth of the pathogens was inhibited completely by 500 ppm of the active compound I-1 or I-2.

1-10. (canceled)

11. A compound of formula I 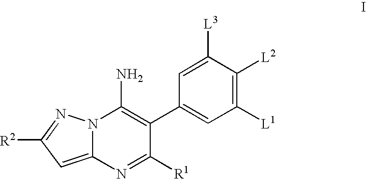
wherein:
L¹ and L³ independently of one another are hydrogen, halogen, hydroxyl, mercapto, nitro, NR^{A}R^{B}, C₁-C₄-haloalkyl, C₁-C₁₀-alkyl, C₂-C₆-alkenyl, C₂-C₆-alkynyl, C₁-C₈-alkoxy, phenyl, phenoxy, phenylthio, benzyloxy or benzylthio;
wherein R^{A} and R^{B} are hydrogen or C₁-C₆-alkyl;
L² is hydrogen, halogen, hydroxyl, mercapto, nitro, NR^{A}R^{B}, C₁-C₄-haloalkyl, CH₂—C₁-C₆-alkyl, C₂-C₆-alkenyl, C₂-C₆-alkynyl, C₁-C₈-alkoxy, phenyl, phenoxy, phenylthio, benzyloxy or benzylthio;
wherein two adjacent groups from the group consisting of L¹, L² and L³ together may be a C₁-C₄-alkylene, C₁-C₄-oxyalkylene, C₁-C₄-oxyalkyleneoxy or butadienyl group;
wherein the groups L¹, L² or L³ are unsubstituted or substituted by one to four identical or different groups R^{a}:
R^{a} is halogen, cyano, hydroxyl, mercapto, C₁-C₁₀-alkyl, C₁-C₁₀-haloalkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl or NR^{A}R^{B};
R¹ is ethyl or n-propyl;
R² is hydrogen, halogen, cyano, NR^{A}R^{B}, hydroxyl, mercapto, C₁-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkenyloxycarbonyl, phenyl, phenoxy, phenylthio, benzyloxy, benzylthio, C₁-C₆-alkyl-S(O)ₘ— or a five- or six-membered saturated, partially unsaturated or aromatic heterocycle which contains one to four heteroatoms from the group consisting of O, N and S;
m is 0, 1 or 2;
wherein the cyclic groups in L¹, L², L³, R^{a} and/or R¹ are unsubstituted or substituted by one to four groups R^{b}:
R^{b} is halogen, cyano, hydroxyl, mercapto, nitro, NR^{A}R^{B}, C₁-C₁₀-alkyl, C₁-C₆-haloalkyl, C₂-C₆-alkenyl, C₂-C₆-alkynyl, C₁-C₆-alkoxy or a five- or six-membered saturated, partially unsaturated or aromatic heterocycle which contains one to four heteroatoms from the group consisting of O, N or S which may be unsubstituted or substituted by one or more halogen and/or C₁-C₄-alkyl groups;
provided that at least one group L¹, L² or L³ is not hydrogen.

12. The compound of claim 11, wherein R¹ is ethyl.

13. The compound of claim 11, wherein R² is methyl or amino.

14. The compound of claim 12, wherein R² is methyl or amino.

15. The compound of claim 11, wherein L³ is hydrogen.

16. The compound of claim 12, wherein L³ is hydrogen.

17. The compound of claim 13, wherein L³ is hydrogen.

18. A process for preparing a compound of formula I 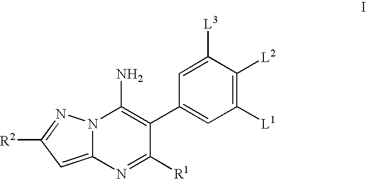
wherein:
L¹ and L³ independently of one another are hydrogen, halogen, hydroxyl, mercapto, nitro, NR^{A}R^{B}, C₁-C₄-haloalkyl, C₁-C₁₀-alkyl, C₂-C₆-alkenyl, C₂-C₆-alkynyl, C₁-C₈-alkoxy, phenyl, phenoxy, phenylthio, benzyloxy or benzylthio;
wherein R^{A} and R^{B} are hydrogen or C₁-C₆-alkyl;
L² is hydrogen, halogen, hydroxyl, mercapto, nitro, NR^{A}R^{B}, C₁-C₄-haloalkyl, CH₂—C₁-C₆-alkyl, C₂-C₆-alkenyl, C₂-C₆-alkynyl, C₁-C₈-alkoxy, phenyl, phenoxy, phenylthio, benzyloxy or benzylthio;
wherein two adjacent groups from the group consisting of L¹, L² and L³ together may be a C₁-C₄-alkylene, C₁-C₄-oxyalkylene, C₁-C₄-oxyalkyleneoxy or butadienyl group;
wherein the groups L¹, L² or L³ are unsubstituted or substituted by one to four identical or different groups R^{a}:
R^{a} is halogen, cyano, hydroxyl, mercapto, C₁-C₁₀-alkyl, C₁-C₁₀-haloalkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl or NR^{A}R^{B};
R¹ is ethyl or n-propyl;
R² is hydrogen, halogen, cyano, NR^{A}R^{B}, hydroxyl, mercapto, C₁-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkenyloxycarbonyl, phenyl, phenoxy, phenylthio, benzyloxy, benzylthio, C₁-C₆-alkyl-S(O)ₘ— or a five- or six-membered saturated, partially unsaturated or aromatic heterocycle which contains one to four heteroatoms from the group consisting of O, N and S;
m is 0, 1 or 2;
wherein the cyclic groups in L¹, L², L³, R^{a} and/or R¹ are unsubstituted or substituted by one to four groups R^{b}:
R^{b} is halogen, cyano, hydroxyl, mercapto, nitro, NR^{A}R^{B}, C₁-C₁₀-alkyl, C₁-C₆-haloalkyl, C₂-C₆-alkenyl, C₂-C₆-alkynyl, C₁-C₆-alkoxy or a five- or six-membered saturated, partially unsaturated or aromatic heterocycle which contains one to four heteroatoms from the group consisting of O, N or S which may be unsubstituted or substituted by one or more halogen and/or C₁-C₄-alkyl groups;
provided that at least one group L¹, L² or L³ is not hydrogen;
comprising
reacting a compound of formula II 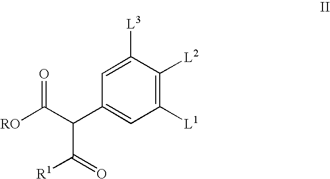
wherein R is C₁-C₄-alkyl and L¹, L², L³ and R¹ are as described above, with a compound of formula III 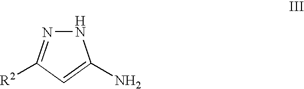
wherein R² is as described above, to give a compound of formula IV, 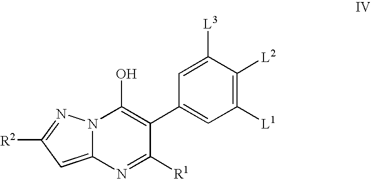
wherein L¹, L², L³, R¹ and R² are as described above;
halogenating the compound of formula IV to give a compound of formula V; 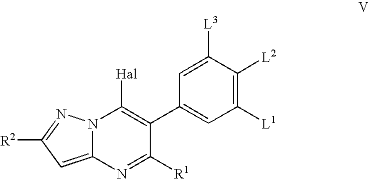
wherein Hal is chlorine or bromine and L¹, L², L³, R¹ and R² are as described above; and
reacting the compound of formula V with ammonia;
wherein a compound of formula I is prepared.

19. A compound of formula IV 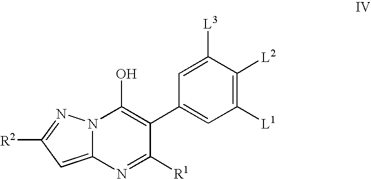
wherein:
L¹ and L³ independently of one another are hydrogen, halogen, hydroxyl, mercapto, nitro, NR^{A}R^{B}, C₁-C₄-haloalkyl, C₁-C₁₀-alkyl, C₂-C₆-alkenyl, C₂-C₆-alkynyl, C₁-C₈-alkoxy, phenyl, phenoxy, phenylthio, benzyloxy or benzylthio;
wherein R^{A} and R^{B} are hydrogen or C₁-C₆-alkyl;
L² is hydrogen, halogen, hydroxyl, mercapto, nitro, NR^{A}R^{B}, C₁-C₄-haloalkyl, CH₂—C₁-C₆-alkyl, C₂-C₆-alkenyl, C₂-C₆-alkynyl, C₁-C₈-alkoxy, phenyl, phenoxy, phenylthio, benzyloxy or benzylthio;
wherein two adjacent groups from the group consisting of L¹, L² and L³ together may be a C₁-C₄-alkylene, C₁-C₄-oxyalkylene, C₁-C₄-oxyalkyleneoxy or butadienyl group;
wherein the groups L¹, L² or L³ are unsubstituted or substituted by one to four identical or different groups R^{a}:
R^{a} is halogen, cyano, hydroxyl, mercapto, C₁-C₁₀-alkyl, C₁-C₁₀-haloalkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl or NR^{A}R^{B};
R¹ is ethyl or n-propyl;
R² is hydrogen, halogen, cyano, NR^{A}R^{B}, hydroxyl, mercapto, C₁-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkenyloxycarbonyl, phenyl, phenoxy, phenylthio, benzyloxy, benzylthio, C₁-C₆-alkyl-S(O)ₘ— or a five- or six-membered saturated, partially unsaturated or aromatic heterocycle which contains one to four heteroatoms from the group consisting of O, N and S;
m is 0, 1 or 2;
wherein the cyclic groups in L¹, L², L³, R^{a} and/or R¹ are unsubstituted or substituted by one to four groups R^{b}:
R^{b} is halogen, cyano, hydroxyl, mercapto, nitro, NR^{A}R^{B}, C₁-C₁₀-alkyl, C₁-C₆-haloalkyl, C₂-C₆-alkenyl, C₂-C₆-alkynyl, C₁-C₆-alkoxy or a five- or six-membered saturated, partially unsaturated or aromatic heterocycle which contains one to four heteroatoms from the group consisting of O, N or S which may be unsubstituted or substituted by one or more halogen and/or C₁-C₄-alkyl groups;
provided that at least one group L¹, L² or L³ is not hydrogen.

20. A compound of formula V 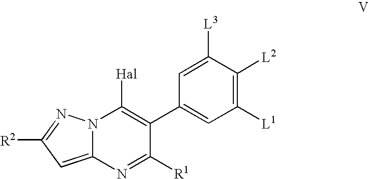
wherein:
Hal is chlorine or bromine;
L¹ and L³ independently of one another are hydrogen, halogen, hydroxyl, mercapto, nitro, NR^{A}R^{B}, C₁-C₄-haloalkyl, C₁-C₁₀-alkyl, C₂-C₆-alkenyl, C₂-C₆-alkynyl, C₁-C₈-alkoxy, phenyl, phenoxy, phenylthio, benzyloxy or benzylthio;
wherein R^{A} and R^{B} are hydrogen or C₁-C₆-alkyl;
L² is hydrogen, halogen, hydroxyl, mercapto, nitro, NR^{A}R^{B}, C₁-C₄-haloalkyl, CH₂—C₁-C₆-alkyl, C₂-C₆-alkenyl, C₂-C₆-alkynyl, C₁-C₈-alkoxy, phenyl, phenoxy, phenylthio, benzyloxy or benzylthio;
wherein two adjacent groups from the group consisting of L¹, L² and L³ together may be a C₁-C₄-alkylene, C₁-C₄-oxyalkylene, C₁-C₄-oxyalkyleneoxy or butadienyl group;
wherein the groups L¹, L² or L³ are unsubstituted or substituted by one to four identical or different groups R^{a}:
R^{a} is halogen, cyano, hydroxyl, mercapto, C₁-C₁₀-alkyl, C₁-C₁₀-haloalkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl or NR^{A}R^{B};
R¹ is ethyl or n-propyl;
R² is hydrogen, halogen, cyano, NR^{A}R^{B}, hydroxyl, mercapto, C₁-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkenyloxycarbonyl, phenyl, phenoxy, phenylthio, benzyloxy, benzylthio, C₁-C₆-alkyl-S(O)ₘ— or a five- or six-membered saturated, partially unsaturated or aromatic heterocycle which contains one to four heteroatoms from the group consisting of O, N and S;
m is 0, 1 or 2;
wherein the cyclic groups in L¹, L², L³, R^{a} and/or R¹ are unsubstituted or substituted by one to four groups R^{b}:
R^{b} is halogen, cyano, hydroxyl, mercapto, nitro, NR^{A}R^{B}, C₁-C₁₀-alkyl, C₁-C₆-haloalkyl, C₂-C₆-alkenyl, C₂-C₆-alkynyl, C₁-C₆-alkoxy or a five- or six-membered saturated, partially unsaturated or aromatic heterocycle which contains one to four heteroatoms from the group consisting of O, N or S which may be unsubstituted or substituted by one or more halogen and/or C₁-C₄-alkyl groups;
provided that at least one group L¹, L² or L³ is not hydrogen.

21. A process for preparing a compound of formula I 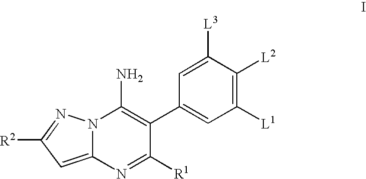
wherein:
L¹ and L³ independently of one another are hydrogen, halogen, hydroxyl, mercapto, nitro, NR^{A}R^{B}, C₁-C₄-haloalkyl, C₁-C₁₀-alkyl, C₂-C₆-alkenyl, C₂-C₆-alkynyl, C₁-C₈-alkoxy, phenyl, phenoxy, phenylthio, benzyloxy or benzylthio;
wherein R^{A} and R^{B} are hydrogen or C₁-C₆-alkyl;
L² is hydrogen, halogen, hydroxyl, mercapto, nitro, NR^{A}R^{B}, C₁-C₄-haloalkyl, CH₂—C₁-C₆-alkyl, C₂-C₆-alkenyl, C₂-C₆-alkynyl, C₁-C₈-alkoxy, phenyl, phenoxy, phenylthio, benzyloxy or benzylthio;
wherein two adjacent groups from the group consisting of L¹, L² and L³ together may be a C₁-C₄-alkylene, C₁-C₄-oxyalkylene, C₁-C₄-oxyalkyleneoxy or butadienyl group;
wherein the groups L¹, L² or L³ are unsubstituted or substituted by one to four identical or different groups R^{a}:
R^{a} is halogen, cyano, hydroxyl, mercapto, C₁-C₁₀-alkyl, C₁-C₁₀-haloalkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl or NR^{A}R^{B};
R¹ is ethyl or n-propyl;
R² is hydrogen, halogen, cyano, NR^{A}R^{B}, hydroxyl, mercapto, C₁-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkenyloxycarbonyl, phenyl, phenoxy, phenylthio, benzyloxy, benzylthio, C₁-C₆-alkyl-S(O)ₘ— or a five- or six-membered saturated, partially unsaturated or aromatic heterocycle which contains one to four heteroatoms from the group consisting of O, N and S;
m is 0, 1 or 2;
wherein the cyclic groups in L¹, L², L³, R^{a} and/or R¹ are unsubstituted or substituted by one to four groups R^{b}:
R^{b} is halogen, cyano, hydroxyl, mercapto, nitro, NR^{A}R^{B}, C₁-C₁₀-alkyl, C₁-C₆-haloalkyl, C₂-C₆-alkenyl, C₂-C₆-alkynyl, C₁-C₆-alkoxy or a five- or six-membered saturated, partially unsaturated or aromatic heterocycle which contains one to four heteroatoms from the group consisting of O, N or S which may be unsubstituted or substituted by one or more halogen and/or C₁-C₄-alkyl groups;
provided that at least one group L¹, L² or L³ is not hydrogen;
comprising
reacting a compound of formula VI, 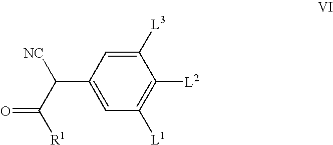
wherein L¹, L², L³ and R¹ are as described above, with a compound of formula III, 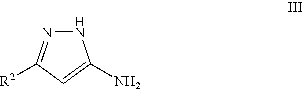
wherein R² is as described above;
wherein a compound of formula I is prepared.

22. A fungicidal composition comprising a solid or liquid carrier and a compound of formula I 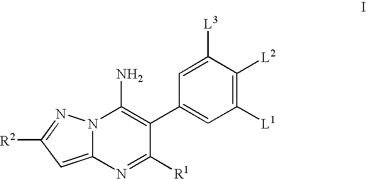
wherein:
L¹ and L³ independently of one another are hydrogen, halogen, hydroxyl, mercapto, nitro NR^{A}R^{B}, C₁-C₄-haloalkyl, C₁-C₁₀-alkyl, C₂-C₆-alkenyl, C₂-C₆-alkynyl, C₁-C₈-alkoxy, phenyl, phenoxy, phenylthio, benzyloxy or benzylthio;
wherein R^{A} and R^{B} are hydrogen or C₁-C₆-alkyl;
L² is hydrogen, halogen, hydroxyl, mercapto, nitro, NR^{A}R^{B}, C₁-C₄-haloalkyl, CH₂—C₁-C₆-alkyl, C₂-C₆-alkenyl, C₂-C₆-alkynyl, C₁-C₈-alkoxy, phenyl, phenoxy, phenylthio, benzyloxy or benzylthio;
wherein two adjacent groups from the group consisting of L¹, L² and L³ together may be a C₁-C₄-alkylene, C₁-C₄-oxyalkylene, C₁-C₄-oxyalkyleneoxy or butadienyl group;
wherein the groups L¹, L² or L³ are unsubstituted or substituted by one to four identical or different groups R^{a}:
R^{a} is halogen, cyano, hydroxyl, mercapto, C₁-C₁₀-alkyl, C₁-C₁₀-haloalkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl or NR^{A}R^{B};
R¹ is ethyl or n-propyl;
R² is hydrogen, halogen, cyano, NR^{A}R^{B}, hydroxyl, mercapto, C₁-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkenyloxycarbonyl, phenyl, phenoxy, phenylthio, benzyloxy, benzylthio, C₁-C₆-alkyl-S(O)ₘ— or a five- or six-membered saturated, partially unsaturated or aromatic heterocycle which contains one to four heteroatoms from the group consisting of O, N and S;
m is 0, 1 or 2;
wherein the cyclic groups in L¹, L², L³, R^{a} and/or R¹ are unsubstituted or substituted by one to four groups R^{b}:
R^{b} is halogen, cyano, hydroxyl, mercapto, nitro, NR^{A}R^{B}, C₁-C₁₀-alkyl, C₁-C₆-haloalkyl, C₂-C₆-alkenyl, C₂-C₆-alkynyl, C₁-C₆-alkoxy or a five- or six-membered saturated, partially unsaturated or aromatic heterocycle which contains one to four heteroatoms from the group consisting of O, N or S which may be unsubstituted or substituted by one or more halogen and/or C₁-C₄-alkyl groups;
provided that at least one group L¹, L² or L³ is not hydrogen.

23. Seed comprising a compound of formula I 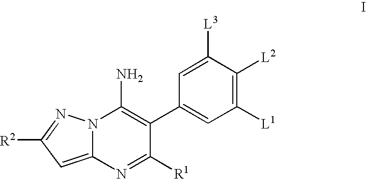
wherein:
L¹ and L³ independently of one another are hydrogen, halogen, hydroxyl, mercapto, nitro, NR^{A}R^{B}, C₁-C₄-haloalkyl, C₁-C₁₀-alkyl, C₂-C₆-alkenyl, C₂-C₆-alkynyl, C₁-C₈-alkoxy, phenyl, phenoxy, phenylthio, benzyloxy or benzylthio;
wherein R^{A} and R^{B} are hydrogen or C₁-C₆-alkyl;
L² is hydrogen, halogen, hydroxyl, mercapto, nitro, NR^{A}R^{B}, C₁-C₄-haloalkyl, CH₂—C₁-C₆-alkyl, C₂-C₆-alkenyl, C₂-C₆-alkynyl, C₁-C₈-alkoxy, phenyl, phenoxy, phenylthio, benzyloxy or benzylthio;
wherein two adjacent groups from the group consisting of L¹, L² and L³ together may be a C₁-C₄-alkylene, C₁-C₄-oxyalkylene, C₁-C₄-oxyalkyleneoxy or butadienyl group;
wherein the groups L¹, L² or L³ are unsubstituted or substituted by one to four identical or different groups R^{a}:
R^{a} is halogen, cyano, hydroxyl, mercapto, C₁-C₁₀-alkyl, C₁-C₁₀-haloalkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl or NR^{A}R^{B};
R¹ is ethyl or n-propyl;
R² is hydrogen, halogen, cyano, NR^{A}R^{B}, hydroxyl, mercapto, C₁-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkenyloxycarbonyl, phenyl, phenoxy, phenylthio, benzyloxy, benzylthio, C₁-C₆-alkyl-S(O)ₘ— or a five- or six-membered saturated, partially unsaturated or aromatic heterocycle which contains one to four heteroatoms from the group consisting of O, N and S;
m is 0, 1 or 2;
wherein the cyclic groups in L¹, L², L³, R^{a} and/or R¹ are unsubstituted or substituted by one to four groups R^{b}:
R^{b} is halogen, cyano, hydroxyl, mercapto, nitro, NR^{A}R^{B}, C₁-C₁₀-alkyl, C₁-C₆-haloalkyl, C₂-C₆-alkenyl, C₂-C₆-alkynyl, C₁-C₆-alkoxy or a five- or six-membered saturated, partially unsaturated or aromatic heterocycle which contains one to four heteroatoms from the group consisting of O, N or S which may be unsubstituted or substituted by one or more halogen and/or C₁-C₄-alkyl groups;
provided that at least one group L¹, L² or L³ is not hydrogen;
in an amount of from 1 g to 1000 g per 100 kg of seed.

24. A method for controlling phytopathogenic harmful fungi comprising
treating fungi or the materials, plants, the soil or seed to be protected from fungal attack with an effective amount of the compound of formula I 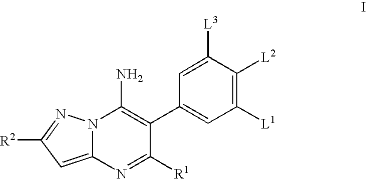
wherein:
L¹ and L³ independently of one another are hydrogen, halogen, hydroxyl, mercapto, nitro, NR^{A}R^{B}, C₁-C₄-haloalkyl, C₁-C₁₀-alkyl, C₂-C₆-alkenyl, C₂-C₆-alkynyl, C₁-C₈-alkoxy, phenyl, phenoxy, phenylthio, benzyloxy or benzylthio;
wherein R^{A} and R^{B} are hydrogen or C₁-C₆-alkyl;
L² is hydrogen, halogen, hydroxyl, mercapto, nitro, NR^{A}R^{B}, C₁-C₄-haloalkyl, CH₂—C₁-C₆-alkyl, C₂-C₆-alkenyl, C₂-C₆-alkynyl, C₁-C₈-alkoxy, phenyl, phenoxy, phenylthio, benzyloxy or benzylthio;
wherein two adjacent groups from the group consisting of L¹, L² and L³ together may be a C₁-C₄-alkylene, C₁-C₄-oxyalkylene, C₁-C₄-oxyalkyleneoxy or butadienyl group;
wherein the groups L¹, L² or L³ are unsubstituted or substituted by one to four identical or different groups R^{a}:
R^{a} is halogen, cyano, hydroxyl, mercapto, C₁-C₁₀-alkyl, C₁-C₁₀-haloalkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl or NR^{A}R^{B};
R¹ is ethyl or n-propyl;
R² is hydrogen, halogen, cyano, NR^{A}R^{B}, hydroxyl, mercapto, C₁-C₆-alkyl, C₁-C₆-haloalkyl, C₃-C₈-cycloalkyl, C₁-C₆-alkoxy, C₁-C₆-alkylthio, C₃-C₈-cycloalkoxy, C₃-C₈-cycloalkylthio, carboxyl, formyl, C₁-C₁₀-alkylcarbonyl, C₁-C₁₀-alkoxycarbonyl, C₂-C₁₀-alkenyloxycarbonyl, C₂-C₁₀-alkenyloxycarbonyl, phenyl, phenoxy, phenylthio, benzyloxy, benzylthio, C₁-C₆-alkyl-S(O)ₘ— or a five- or six-membered saturated, partially unsaturated or aromatic heterocycle which contains one to four heteroatoms from the group consisting of O, N and S;
m is 0, 1 or 2;
wherein the cyclic groups in L¹, L², L³, R^{a} and/or R¹ are unsubstituted or substituted by one to four groups R^{b}:
R^{b} is halogen, cyano, hydroxyl, mercapto, nitro, NR^{A}R^{B}, C₁-C₁₀-alkyl, C₁-C₆-haloalkyl, C₂-C₆-alkenyl, C₂-C₆-alkynyl, C₁-C₆-alkoxy or a five- or six-membered saturated, partially unsaturated or aromatic heterocycle which contains one to four heteroatoms from the group consisting of O, N or S which may be unsubstituted or substituted by one or more halogen and/or C₁-C₄-alkyl groups;
provided that at least one group L¹, L² or L³ is not hydrogen;
wherein the phytopathogenic harmful fungi are controlled.
